(19) United States
(12) Patent Application Publication
(10) Pub. No.: US 2026/0183129 A1
(43) Pub. Date: Jul. 2, 2026

(54) SYSTEMS AND METHODS FOR FABRICATING A PROSTHETIC SOCKET

(71) Applicant: Michael P. SMITH, Tamuning, GU (US)

(72) Inventor: Michael P. SMITH, Tamuning, GU (US)

(21) Appl. No.: 19/424,437

(22) Filed: Dec. 18, 2025

Related U.S. Application Data
(60) Provisional application No. 63/738,987, filed on Dec. 26, 2024.

(51) Int. Cl. A61F 2/50

(52) U.S. Cl. CPC A61F 2/5046; A61F 2002/5052

(57) ABSTRACT

A method of fabricating a prosthesis socket includes applying a socket material to a portion of a limb. After applying the socket material, the portion of the limb is placed into an interior region of a flexible liner of a socket fabrication system, which interior region also includes a set of beads. The socket fabrication system includes a rigid outer cylinder defining an interior volume in which the flexible liner is disposed. A top end portion of the flexible liner is sealed to an upper portion of the limb positioned outside of the cylinder and flexible liner. A vacuum is produced within the interior region of the flexible liner with the patient in a standing position and the rigid outer cylinder bearing a portion of a weight of the patient. The vacuum causes the plurality of beads to compress the socket material to conform to the limb.

### CROSS-REFERENCE TO RELATED APPLICATIONS

This application claims benefit of priority to U.S. Provisional Application No. 63/738,987, entitled “Systems and Methods for Fabricating A Prosthetic Socket,” filed Dec. 26, 2024, which is incorporated herein by reference in its entirety.

### BACKGROUND

The embodiments described herein relate generally to devices used in conjunction with the fabrication of a socket and, more specifically, to a custom socket for attaching a prosthesis or an orthotic to a limb of a patient.

Known methods of fabricating a custom socket for attaching a prosthesis or orthotic to a limb of patient (e.g., to a limb of an amputee) typically require the formation of a mold of the patient's residual limb to use for fabrication of a socket, and then a second process to form the socket about the mold. Such processes can be complicated and time consuming and require the work of an experienced individual with knowledge of such processes. Such processes also require a fabrication facility capable of performing the fabrication. Thus, such facilities for fabricating a socket are limited making it difficult for those in need to have access to fabrication of a custom socket. Accordingly, many known methods require that the patient first visit the practitioner for measurements to make the mold followed by a second visit for the socket (formed from the mold) to be adjusted and fit to the patient's residual limb.

In addition, such processes involve the creation of plaster molds, which can pose health and safety issues for the individuals involved in the fabrication process. For example, known methods can result in inhalation of plaster dust and inhaling of chemical fumes from the resins used in traditional lamination processes.

Some known custom fabrication processes use a water-based process that includes a chamber filled with pressurized water. The limb of the patient (e.g., amputee) is first wrapped with a plaster cast and then the limb is placed in the chamber. Water is pumped into the chamber until the chamber is filled, with the limb inside the chamber. The water pressure forms the cast to the limb forming a cast mold or a socket. Such water-based processes have several disadvantages. First, by forming the cast mold in water, the pressure applied is static as the plaster hardens. Second, by filling the chamber with water the device is pushing against the limb within the chamber which can push the limb up and out of the chamber. In the case of fabricating a cast mold formed over the limb in the water, the plaster cast mold (e.g., negative mold) then must be filled with plaster of Paris to form a positive mold the patient's limb, again exposing the individual fabricating the socket to a plater-filled environment. After the positive mold is modified to fit the individual patient, the modified positive mold must then have the custom socket laminated over the positive mold.

This, a need exists for an improved custom fabrication system and process for fabricating a custom socket for attaching a prosthesis or orthotic to a limb of a patient.

### SUMMARY

Devices and methods for the fabrication of a socket and, more specifically, to a custom socket for attaching a prosthesis or an orthotic to a limb of a patient are described herein. In some embodiments, an apparatus includes a support stand and a rigid outer cylinder coupled to the support stand. The outer cylinder defines an interior volume and an opening that provides access to the interior volume. The apparatus further includes a flexible liner configured to be disposed inside the interior volume of the outer cylinder. The flexible liner defines an interior region and a limb opening at a top end of the flexible liner. The limb opening of the flexible liner is configured to receive a portion of a limb of a patient with a socket material disposed thereon such that the portion of the limb with the socket material thereon is positioned inside the interior region of the flexible liner. The top end of the flexible liner is configured to be sealed around the limb at an upper portion of the limb. The apparatus further includes a plurality of beads disposed within the interior region of the flexible liner and a vacuum connector having a first end and a second end. The first end of the vacuum connector is coupled to a bottom end of the outer cylinder and in fluid communication with the interior region of the flexible liner, and the second end of the vacuum connector is configured to be coupled to a vacuum pump configured to create a vacuum within the flexible liner such that the plurality of beads compress against the socket material and the portion of the limb when the portion of the limb is positioned in the interior region of the flexible liner and the top end of the flexible liner is sealed to the limb.

In some embodiments, a method of fabricating a socket for attaching a prosthesis or an orthotic to a limb includes applying a socket material to a portion of a limb of a patient. The socket material is used in the formation of the socket. After applying the socket material, the portion of the limb is placed into an interior region of a flexible liner of a socket fabrication system. The socket fabrication system includes a rigid outer cylinder defining an interior volume in which the flexible liner is configured to be disposed. A plurality of beads is within the interior region of the flexible liner. A top end portion of the flexible liner is sealed to an upper portion of the limb positioned outside of the cylinder and flexible liner. A vacuum is produced within the interior region of the flexible liner with the patient in a standing position and the rigid outer cylinder bearing a portion of a weight of the patient. The vacuum causes the plurality of beads to compress against the socket material such that the socket material conforms to the portion of the limb.

In some embodiments, a kit for fabricating a socket for attaching a prosthesis or an orthotic to a limb using a socket fabrication system includes a flexible liner, a plurality of beads, a protective liner, a socket material, and a cover. The flexible liner is configured to be disposed inside an interior volume of a rigid outer cylinder of the socket fabrication system. The flexible liner defines an interior region configured to receive an end portion of a limb of a patient with a socket material disposed thereon. The flexible liner further includes a coupling configured to be coupled to a vacuum pump via a vacuum hose to place the vacuum pump in fluid communication with the interior region of the flexible liner. The plurality of beads is configured to be disposed within the interior region of the flexible liner. The protective liner is configured to be placed on the end portion of the limb of the patient. The socket material is configured to be applied over the protective liner and the cover is configured to be placed over the socket material.

### BRIEF DESCRIPTION OF THE DRAWINGS

FIG. 1A is a schematic illustration of a socket fabrication system, according to an embodiment.

FIG. 1B is a schematic illustration of a residual limb of a patient for which a socket can be formed via the socket fabrication system of FIG. 1A.

FIG. 1C is a schematic illustration of the socket fabrication system of FIG. 1A with additional components added to the system, and illustrating a residual limb of a patient disposed within the socket fabrication system.

FIG. 2 is a flowchart illustrating a method of fabricating a custom socket for attaching a prosthesis or an orthotic to a limb of a patient.

FIG. 3A is a schematic illustration of a socket fabrication system according to an embodiment, shown during a fabrication process with a limb of a patient.

FIG. 3B is an enlarged view of the encircled portion B in FIG. 3A.

FIG. 4 is a schematic illustration of an embodiment of a socket formed with the socket fabrication system of FIG. 3A shown coupled to a residual limb of a patient.

FIG. 5 is a schematic illustration of an embodiment of a socket formed with the socket fabrication system of FIG. 3A, shown coupled to a residual limb of a patient.

FIG. 6 is a schematic illustration of an embodiment of a socket formed with the socket fabrication system of FIG. 3A, shown coupled to a residual limb of a patient.

FIG. 7 is a schematic illustration of a flexible liner of a socket fabrication system, according to an embodiment.

FIG. 8 is a schematic illustration of a portion of the flexible liner of FIG. 8 coupled to a portion of a socket fabrication system.

FIG. 9 is an illustration of a socket fabrication system according to another embodiment, shown during a fabrication process with a limb of a patient.

FIG. 10 is a schematic illustration of a kit including components used in the fabrication of a socket for attaching a prosthetic or an orthotic to a limb of a patient.

### DETAILED DESCRIPTION

Systems, devices and methods are described herein for the custom fabrication of a socket for attaching a prosthetic or orthotic to a limb of a patient. The custom fabrication process provides for formation of the socket directly to the limb of the patient. Thus, the system and methods described herein eliminate the need for the multi-step process involving fabricating a negative mold, a positive mold, and then forming the socket about the positive mold.

By eliminating two steps of a typical socket fabrication process, the time to fabricate a custom prosthesis or orthotic can be reduced. In some circumstances, the systems and methods described herein allow for the patient to receive a custom-molded socket in a single visit. Additionally, the systems and methods described herein can reduce the experience and knowledge needed by an individual to learn the fabrication process. This will allow for more individuals in need of a custom socket to receive the proper prosthetic care they need. It will also reduce the size of a fabrication facility needed to make a custom socket. By directly forming the socket to the individual, the need for plaster modification rooms and lamination rooms will be reduced or eliminated.

The fabrication systems and methods described herein can also improve the health and safety of individuals involved in the prosthetic socket fabrication process. By eliminating the need for plaster molds, the individuals involved in the fabrication of the socket will no longer inhale plaster dust. Further, lamination of the custom socket will no longer be needed eliminating inhalation of chemical fumes from the resins used in traditional lamination processes. Thus, the air quality in socket fabrication facilities can be improved and fewer wasted materials that are normally discarded during traditional socket fabrication processes.

By simplifying the process, the knowledge and experience needed for the fabricator, and reducing the needs of the fabrication facility, more individuals can be trained to perform the fabrication, more facilities can be utilized, resulting in the expansion of available care for patients in need of a prosthetic socket.

As described herein, fabrication systems and methods are provided that use a vacuum force to form (or mold) the socket directly to the limb of the patient. Beads are placed within a chamber of the system and vacuum is applied such that the beads conform to the limb of the patient. The vacuum force increases pressure on soft tissue and does not compress around the limb of the patient, but instead will move around the bony prominences in the limb. The vacuum pressure is distributed evenly around the limb and will not push or pull the limb as is possible in a pressurized system. The beads within the chamber will compress evenly around the limb. Thus, the socket is molded directly to the limb of the patient without the limb being unnaturally deformed in a manner that would disrupt the fit between the socket and limb during normal use.

The systems and processes described herein can also be used to form a cast mold of a residual limb of a patient, or a cast mold of a patient's extremities (e.g., foot, hand), or a custom molded device directly to the patient's extremity. For example, the system can be used to fabricate a cast mold of a limb, which can then be filled with plaster of Paris and a custom socket can be formed directly over it using conventional methods.

In some embodiments, a fabrication system and method described herein can be used to fabricate a custom-molded prosthetic socket directly to a limb of a patient (e.g., an amputee's residual limb). The fabrication system includes a rigid outer cylinder having an interior volume. The outer cylinder is coupled to an adjustable stand that allows the cylinder to be moved up and down to position the cylinder to meet the height of the patient. A flexible liner defining an interior region is placed inside the interior volume of the rigid outer cylinder. Multiple beads are within, or placed within, the interior region of the flexible liner. A vacuum connector is coupled at one end to a bottom end of the flexible liner and at the other end to a vacuum pump.

The process of forming the custom socket directly to the patient's limb includes adjusting the height of the cylinder to correspond to the height of the patient when the patient is standing such that a portion of the limb of the patient can be positioned within the cylinder when the patient is in a standing position. The patient's limb is then prepped for the process with the following socket material components being applied to the patient's limb. First, a protective liner is applied to the limb. The protective liner can be of any suitable liner for orthotic use, such as, for example, a gel material and can have various thicknesses. For example, the protective liner can be formed with a silicone material. Second, a socket material, including, for example, a fiber material and a hardenable material impregnated in or coupled to the fiber material is applied over the protective liner. For example, the socket material can be in the form of a roll on sock. The socket material is then wetted with, for example, water to start a chemical reaction that will allow the socket material (e.g., fiber and hardenable material) to harden over a set period of time. Next, a thin plastic cover or sleeve is placed over the wetted socket material so that the beads in the flexible liner of the fabrication system will not stick to the socket fiber material.

After the socket material components are applied to the limb, the patient will then stand and insert their limb (with the socket material thereon and covered by the plastic cover) into the beads in the interior region of the flexible liner. A top end portion of the flexible liner is then sealed to an upper portion of the limb outside of the cylinder. The patient will stand on their limb within the cylinder such that the patient is standing evenly on both the patient's limb within the cylinder and the patient's other limb on the support surface (e.g., floor). The vacuum pump is turned on and a vacuum will be applied to the patient's limb within the cylinder and over the socket material on the patient's limb. The patient will stay in this position with the vacuum actuated for a time period sufficient for the socket material to harden. The patient will then remove the limb from the cylinder with the now hardened socket formed on the limb. The hardened socket is then removed from the limb and can be cut, trimmed and finished as needed. The custom socket can then be attached to other socket components needed to attach the socket to a prosthesis or orthotic. For example, the socket can be coupled to an adapter to attach to the prosthesis or orthotic.

As used herein, the term “limb” means an appendage of a patient including a residual limb of an amputee (e.g., residual limb of an amputated leg), or an extremity of a patient, such as a hand or a foot of a patient.

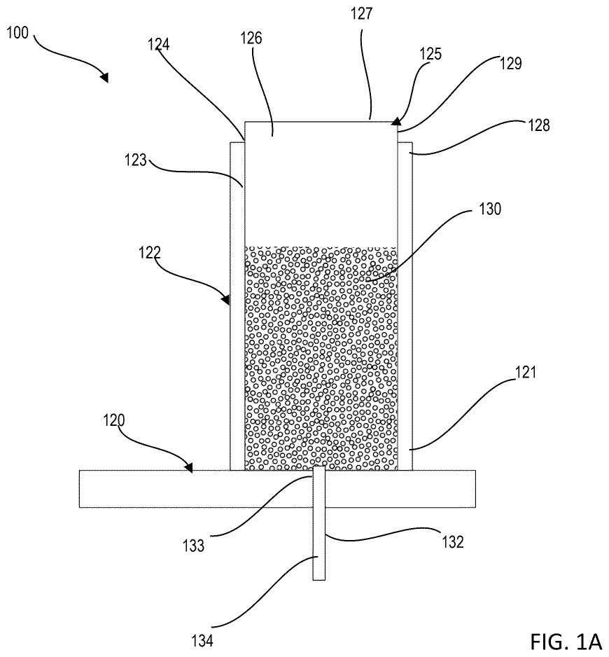

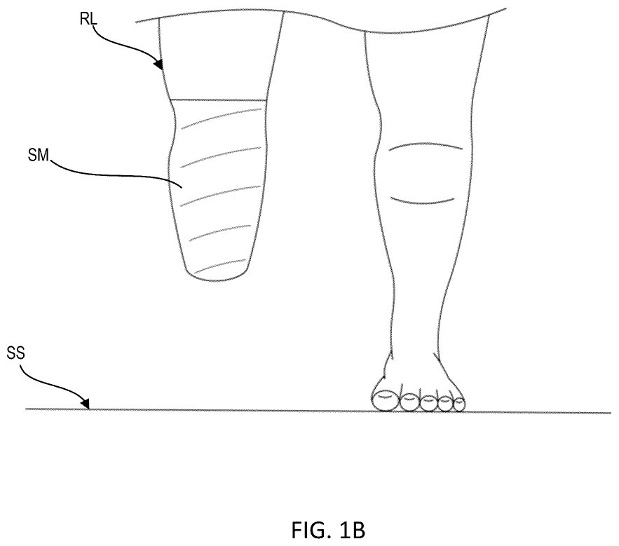

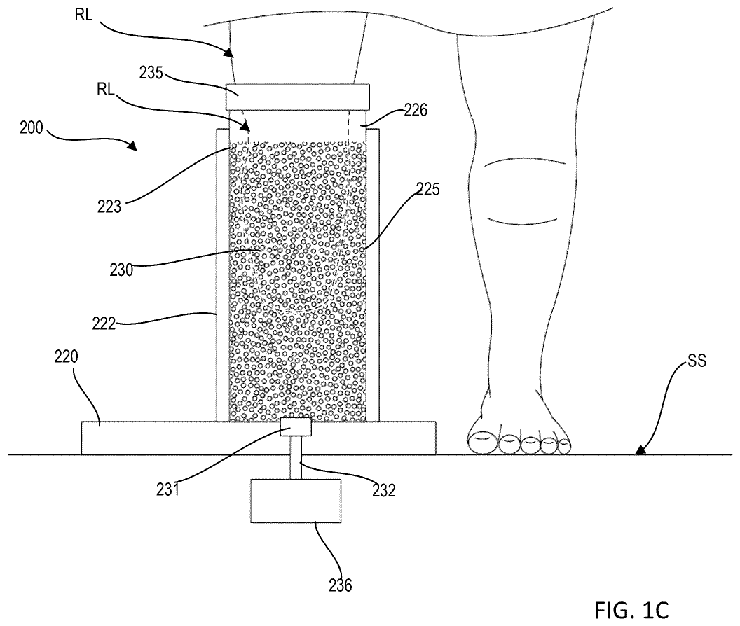

FIG. 1A is a schematic illustration of an embodiment of a socket fabrication system that can be used to fabricate a custom socket to attach a prosthesis or orthotic to a limb of a patient. The socket fabrication system 100 includes a support stand 120 and a rigid outer cylinder 122 coupled to the support stand 120. The outer cylinder 122 defines an interior volume 123 and an opening 124 at a top end 128 of the outer cylinder 122 that provides access to the interior volume 123. The outer cylinder 122 is sized and configured to receive a portion of the patient's limb. Moreover, the outer cylinder 122 and the stand 120 are configured to support at least a portion of the patient's weight. Thus, the outer cylinder 122 and the stand 120 can be constructed from any material having suitable strength, such as, for example, mild steel, aluminum alloy, or a rigid composite material. A flexible liner 125 is configured to be disposed inside the interior volume 123 of the outer cylinder 122. The flexible liner 125 can be constructed with, for example, a rubber, plastic or other flexible material. The flexible liner 125 defines an interior region 126 and a limb opening 127 at a top end 129 of the flexible liner 125, and multiple beads 130 are disposed within the interior region 126 of the flexible liner 125. The beads 130 can be of any suitable size to be compressed about a portion of the patient's limb during use, thereby allowing the socket material to be formed tightly to the patient's limb. For example, in some embodiments, the beads 130 can be between about 1 mm and 15 mm in diameter. In other embodiments, the beads 130 can be between about 4 mm and 10 mm in diameter. In yet other embodiments, the beads need not be spherical but can be of any desired shape. The beads 130 can be formed with any suitable material, for example, closed-cell extruded polystyrene foam (Styrofoam™), rice, BB's, or air gun pellets.

The limb opening 127 of the flexible liner 125 is configured to receive a portion of a limb (see, e.g., residual limb RL in the example of FIG. 1B) of a patient prepped with a protective liner and a socket material SM (see FIG. 1B) disposed thereon. The protective liner can be any suitable liner for orthotic use. For example, in some embodiments, the protective liner is a gel liner. In some embodiments, the protective liner can be formed with, for example, a thermoplastic elastomer or a silicone material and can have suitable thickness and properties to function as the interface between the residual limb and the socket produced by the system 100. In some embodiments, the protective liner can include antioxidants that can be released from the protective liner over time (e.g., Vitamins A, B and C or any other antioxidants), skin conditioning agents (e.g., mineral oil or baby oil), or medicaments (e.g., to limit infection, promote healing of sores, etc.). In some embodiments, the protective liner can include multiple materials, such as an interior thermoplastic elastomer with an outer fabric layer. In some embodiments, the protective liner can include an attachment member, such as a pin (e.g., see pin 657 below), a stud or an internally threaded boss, to which a mating structure of an orthotic can be coupled.

The socket material SM can be formed with multiple components as described herein. For example, the socket material SM can include a fiber material that is impregnated with a hardenable material or the hardenable material can be otherwise coupled to the fiber material. For example, the socket material SM can include, for example, a fiber material that is impregnated with a resin, such as polyurethane. In other embodiments the fiber material can be impregnated with a fiber-reinforced thermoplastic. In some embodiments, the socket material can be formed with a heat-moldable material. The socket material can be applied to the limb as a wrap or a “sock” that is placed over or wrapped about the gel liner. In use, socket material is wetted with, for example, water to activate the hardenable material, and then a cover is placed over the socket material. The cover can be, for example, a thin plastic sheet of material wrapped around the socket material on the limb of the patient. In some embodiments, the cover can be in the form of a sleeve.

The limb of the patient can be, for example, a residual limb of an amputee, such as a residual end of a leg of a patient as shown in FIG. 1B. The limb can also be an extremity of a patient, such as a hand or a foot. The limb can be disposed within the interior region 126 of the flexible liner 125 such that the portion of the limb with the socket material thereon is positioned inside the interior region 126 of the flexible liner 125 and an upper portion of the limb is positioned outside of the interior region 126. The top end 129 of the flexible liner 125 is configured to be sealed around the limb at a location on the upper portion of the limb. For example, the flexible liner 125 can be sealed to the limb using a sealing member, such as a tape, an elastomeric band, an adhesive liner, a belt, or other sealing member that can be sealed during the fabrication process and then removed after the process using the socket fabrication process is completed. FIG. 1C illustrates an example of a sealing member 235. In some embodiments, sealing a top end portion of the flexible liner 125 includes maintaining the top end portion of the flexible liner 125 free from any direct coupling to the outer cylinder 122. In this manner, the top end portion of the flexible liner 125 with the limb RL therein is not coupled to the outer cylinder 122. This arrangement allows for shifting of the limb RL within the outer cylinder 122 (e.g., as a result of the patient shifting weight between limbs) without impacting the seal produced by the sealing member 235. Similarly stated, the arrangement advantageously allows the residual limb RL to move within the outer cylinder 122 while maintaining the seal between the residual limb RL and the flexible liner 125.

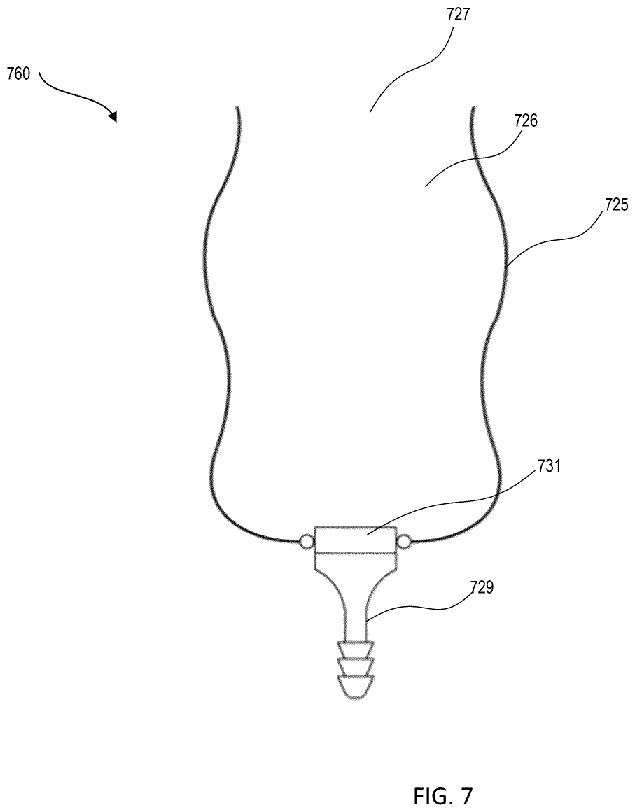

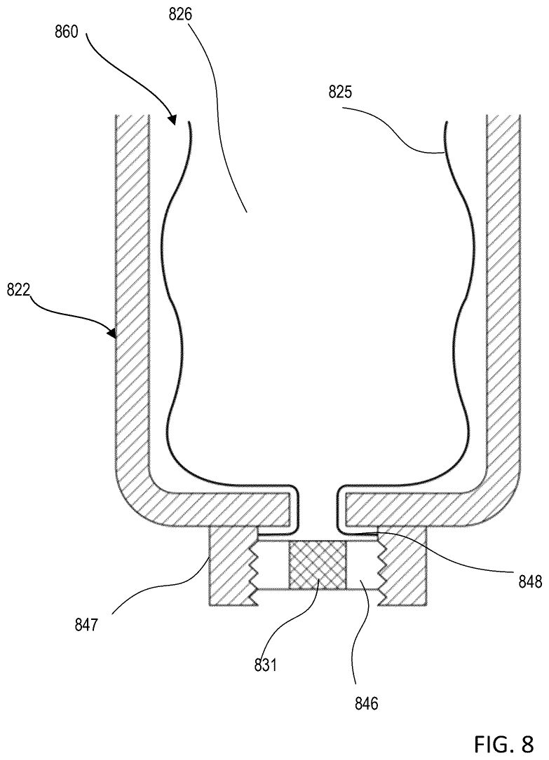

A vacuum connector 132 has a first end 133 coupled to a bottom end 121 of the outer cylinder 122 and in fluid communication with the interior region 126 of the flexible liner 125. For example, the flexible liner 125 can include a connector (not shown in FIG. 1A) at a bottom end portion that couples to a connector or other component at the bottom end 121 of the outer cylinder 122. In some embodiments the connector on the flexible liner 125 can extend through an opening in the outer cylinder 122. In some embodiments, the first end 133 of the vacuum connector 132 can extend through an opening in the bottom of the outer cylinder 122 and be coupled to a connector (not shown) of the flexible liner 125. In this manner, the vacuum connector 132 can be movably coupled to the outer cylinder. In some embodiments, a filter (shown in FIG. 1C) is coupled to the first end 133 of the vacuum connector 132 to prevent the beads 130 from moving out of the interior region 126 of the flexible liner 125 through the vacuum connector 132. FIGS. 7 and 8 described in more detail below illustrate example couplings between the vacuum connector 132 and the flexible liner 125.

The vacuum connector 132 has a second end 134 configured to be coupled to a vacuum pump or other suitable vacuum source (not shown in FIG. 1A). The vacuum connector 132 can include a hose or other tubular structure having a fluid channel to communicate a fluid between the interior region 126 of the flexible liner 125 and the vacuum pump. In some embodiments, the vacuum connector 132 is configured to be coupled to a separate hose or other tubular structure. The vacuum pump is configured to create a vacuum within the interior region 126 of the flexible liner 125 such that the multiple beads 130 compress against the socket material and the portion of the limb disposed within the interior region 126 of the flexible liner 125. For example, in use, the patient while in a standing position on a support surface SS, places their limb with the socket material SM thereon (see FIG. 1B, the residual limb RL) within flexible liner 125 within the cylinder 122 such that the limb is surrounded with the multiple beads 130 inside the flexible liner 125. In some embodiments, the support stand 120 includes an adjustment mechanism (not shown in FIGS. 1A and 1B) configured to adjust a height position of the outer cylinder 122 to correspond to a height of the limb of the patient when the portion of the limb is placed in the flexible liner 125. With the limb of the patient within the rigid cylinder 122, the rigid outer cylinder 122 and/or the stand 120 bears a portion of a weight of the patient during the fabricating process. A vacuum is then created within the flexible liner 125 which causes the beads 130 to compress against the socket material SM such that the socket material SM conforms to the portion of the limb (e.g., residual limb RL) to form a custom socket to attach a prosthesis or orthotic to a limb of a patient. By compressing the socket material SM against the portion of the limb RL using the beads 130, the socket material SM can be conformed to contours and shape of the limb RL so that the resulting socket will fit snugly to the limb without undesired slipping or without causing undesired deformation of the limb. Compressing the socket material SM against the portion of the limb RL using a vacuum limits or eliminates the likelihood that the compression force will push the residual limb RL out of the socket material SM or away from the outer cylinder 122. The vacuum produced can be any suitable value, such as for example between 375 mm Hg and 635 mm Hg (15 inches Hg and 25 inch) or about 500 mm Hg (about 20 inches Hg).

FIG. 1C is a schematic illustration of the socket fabrication system 200 that can be used to fabricate a custom socket to attach a prosthesis or orthotic to a limb of a patient. The socket fabrication system 200 can include all the same components as described above for socket fabrication system 100 along with certain additional components as described herein. Specifically, the socket fabrication system 200 includes a support stand 220 and a rigid outer cylinder 222 coupled to the support stand 220. The outer cylinder 222 includes an interior volume 223 in which a flexible liner 225 can be disposed, and multiple beads 230 are within an interior region 226 of the flexible liner 225. A vacuum connector 232 has a first end 233 coupled to the cylinder 222 and a second end 234 couplable to a vacuum pump 236. Each of these components can be constructed the same as or similar to and perform the same as or similar function as like components described above for socket fabrication system 100. FIG. 1C illustrates the addition of a sealing member 235 at a top end of the flexible liner 225 that can be used to seal the flexible liner 225 to the limb of the patient as described herein. As shown in FIG. 1C, the flexible liner 225 is sealed to the limb of the patient using the sealing member 235 such that here is no direct coupling to a top portion of the outer cylinder 222. The sealing member 235 can be, for example, an elastomeric band, an adhesive liner, a tape, a belt, or other device that can be used to seal the flexible liner around the limb of the patient. Sealing the flexible liner 225 to the limb allows for the vacuum to be maintained within the flexible liner 225 during use. Moreover, as described above, by maintaining the top part of the flexible liner 225 and/or the sealing member 235 without a direct coupling to the outer cylinder 222 allows the residual limb RL to move within the outer cylinder 222 while maintaining the seal between the residual limb RL and the flexible liner 225. FIG. 1C also illustrates a filter 231 that is coupled to the first end 233 of the vacuum connector 232 to prevent the beads 230 from moving out of the interior region 226 of the flexible liner 225 through the vacuum connector 232.

As described herein the vacuum pump 236 can be actuated to create a vacuum within an interior region of the flexible liner 225 such that the multiple beads 230 compress against a portion of a limb of a patient with a socket material disposed thereon (see e.g., limb RL with socket material SM and other fabrication components as shown in FIG. 1B) within the interior region 226 of the flexible liner 225. As described above, the socket material SM can be formed with multiple components as described herein. For example, the socket material SM can include a fiber material that is impregnated with a hardenable material or the hardenable material can be otherwise coupled to the fiber material. For example, the socket material SM can include, for example, a fiber material that is impregnated with a resin, such as polyurethane. In other embodiments the fiber material can be impregnated with a fiber-reinforced thermoplastic. In some embodiments, the socket material can be formed with a heat-moldable material. The socket material can be applied to the limb as a wrap or a “sock” that is placed over or wrapped about the gel liner. In use, socket material is wetted with, for example, water to activate the hardenable material, and then a cover is placed over the socket material. The cover can be, for example, a thin plastic sheet of material wrapped around the socket material on the limb of the patient. In some embodiments, the cover can be in the form of a sleeve.

As described above, a protective liner can be placed over the limb of the patient prior to applying the socket material SM. The protective liner can be any suitable liner for orthotic use. For example, in some embodiments, the protective liner is a gel liner. In some embodiments, the protective liner can be formed with, for example, a thermoplastic elastomer or a silicone material and can have suitable thickness and properties to function as the interface between the residual limb and the socket produced by the fabrication system 200. In some embodiments, the protective liner can include antioxidants that can be released from the protective liner over time (e.g., Vitamins A, B and C or any other antioxidants), skin conditioning agents (e.g., mineral oil or baby oil), or medicaments (e.g., to limit infection, promote healing of sores, etc.). In some embodiments, the protective liner can include multiple materials, such as an interior thermoplastic elastomer with an outer fabric layer. In some embodiments, the protective liner can include an attachment member, such as a pin (e.g., see pin 657 below), a stud or an internally threaded boss, to which a mating structure of an orthotic can be coupled.

In one example use, the patient while in a standing position on a support surface SS, places their limb (see FIG. 1C, the residual limb RL) within the interior region 226 of the flexible liner 225 such that the limb is surrounded with the beads 230. A top end of the flexible liner 225 is sealed around an upper portion of the limb with the seal member 235. flexible liner 225 can be disposed within an interior volume 223 of the cylinder 222 when the limb is placed therein, or the limb with the flexible liner 225 attached thereto can be placed within the interior volume 223 of the cylinder 222. The vacuum 236 is actuated to create a vacuum within the flexible liner 225 which causes the beads 230 to compress against the socket material SM within the flexible liner 225, such that the socket material SM conforms to the portion of the limb (e.g., residual limb RL) to form a custom socket to attach a prosthesis or orthotic to a limb of a patient. The vacuum is actuated for a sufficient amount of time and at a sufficient vacuum pressure (as described above) to allow the socket material to cure and harden on the portion of the limb.

In some embodiments, the support stand 220 can optionally include an adjustment mechanism (not shown in FIG. 1C) configured to adjust a height position of the outer cylinder 222 to correspond to a height of the limb of the patient when the portion of the limb is placed in the flexible liner 225. Thus, with the limb of the patient within the rigid cylinder 222, the rigid outer cylinder 222 bears a portion of a weight of the patient during the fabricating process. In this manner, the system 200 can produce the socket with the residual limb RL in a mid-stance position. By doing so, the residual limb RL is in a natural, weight bearing condition, which causes the skin and/or soft tissue to move slightly relative to the bone as compared to when the residual limb RL is not in a weight bearing condition. This arrangement allows the system to produce a socket under conditions that more accurately reflect the actual conditions of use. Thus, the system 200 and methods produce a socket that will optimally fit the residual limb RL during use.

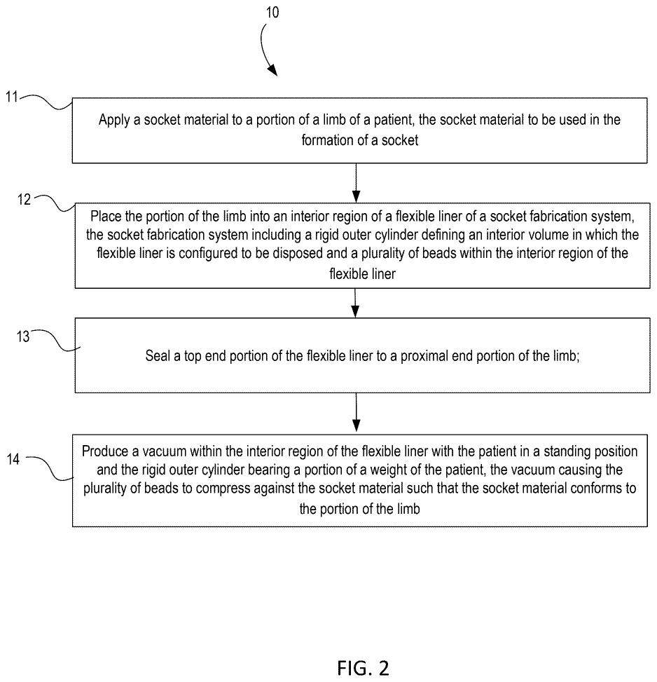

FIG. 2 is a flowchart illustrating a method of fabricating a custom socket for attaching a prosthetic or orthotic to a limb of a patient. The method 10 is described with reference to the socket fabrication system 100 but it should be understood that the method can be performed using any of the embodiments of a socket fabrication system described herein. The method 10 includes at 11, applying a socket material to a portion of a limb of a patient. The socket material is used in the formation of a custom socket. In some embodiments, a protective liner (as described herein) if first placed about the portion of the limb of the patient and then the socket material is applied over the protective liner. The socket material is wetted and a cover is placed over the socket material. The cover can be, for example, a thin plastic sheet of material wrapped around the fiber material on the limb of the patient. In some embodiments, the cover can be in the form of a sleeve.

At 12, after applying the socket material (and other fabrication components) to the limb, the portion of the limb is placed into an interior region of a flexible liner of a socket fabrication system. The socket fabrication system can include any of the components and functions as described herein for any of the embodiments of a socket fabrication system. For example, the socket fabrication system can include a rigid outer cylinder defining an interior volume in which the flexible liner is configured to be disposed, and multiple beads disposed within the interior region of the flexible liner. At 13, a top end portion of the flexible liner is sealed to an upper portion of the limb of the patient outside of the cylinder. For example, as described herein, a sealing member, such an elastomeric band, an adhesive liner, a tape, a belt, or other device that can be used to seal the flexible liner around the limb of the patient, such as, for example, a strap, tape, belt or other suitable component or device that can be used to seal the flexible liner 225 to the limb of the patient. At 14, a vacuum is produced within the interior region of the flexible liner with the patient in a standing position and the rigid outer cylinder bearing a portion of a weight of the patient as shown, for example, in FIGS. 1B and 1C. The vacuum pressure causes the multiple beads to compress against the socket material on the end portion of the limb such that the socket material conforms to the portion of the limb. The vacuum pressure can be any suitable value, such as for example between 375 mm Hg and 635 mm Hg (15 inches Hg and 25 inch) or about 500 mm Hg (about 20 inches Hg). For example, a vacuum connector (e.g., vacuum connector 132) can be coupled at a first end to the outer cylinder 122 and in communication with the interior region 126 of the flexible liner 125. FIGS. 7 and 8 described below illustrate example coupling methods for coupling a flexible liner to the vacuum connector 132.

A second end of the vacuum connector 132 can be coupled to a vacuum pump (e.g., vacuum pump 236). The vacuum pump can be actuated to produce the vacuum pressure within the interior region 126 of the flexible liner 125. In some embodiments, the vacuum pump is actuated for a time period sufficient for the socket material to harden on the portion of the limb.

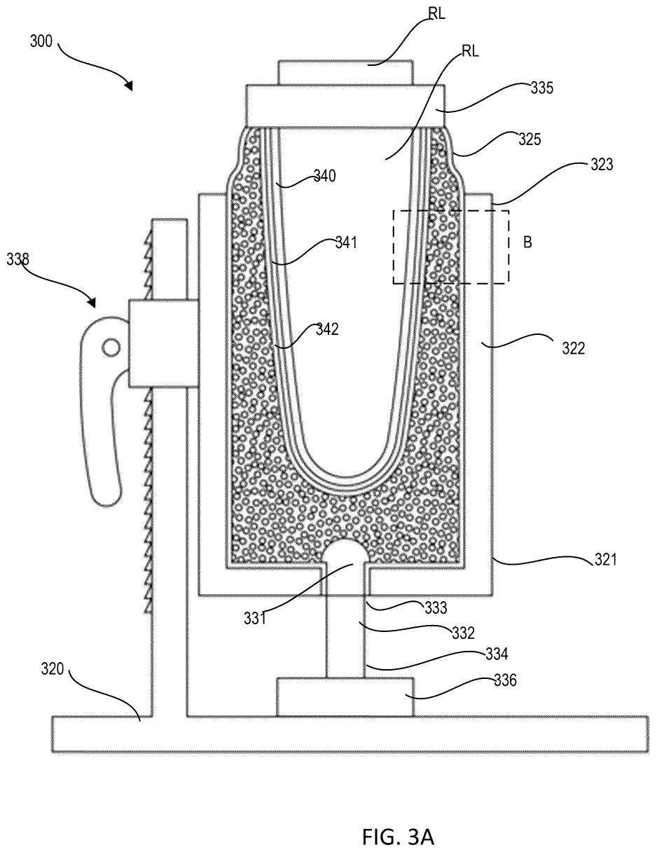

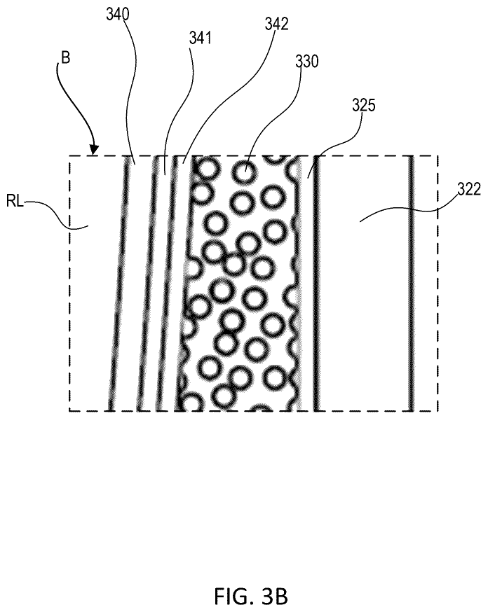

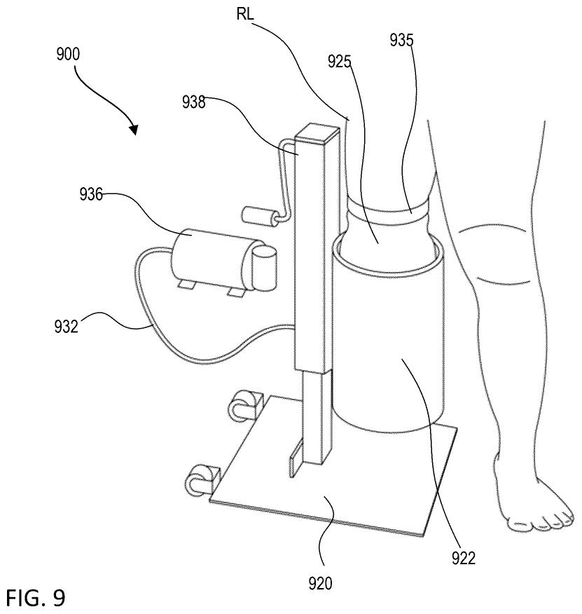

In some embodiments, the socket fabrication system includes a support stand (e.g., 120, 220, 320) including a height adjustment mechanism (e.g., as shown in FIGS. 3A and 9). In such an embodiment, prior to producing a vacuum within the interior region of the flexible liner, a height of the rigid outer cylinder is adjusted to a height associated with a height of the portion of the limb of the patient when the end portion of the limb is placed into the interior region of the flexible liner. In this manner, the method can produce the socket with the residual limb RL in a mid-stance position that is customized to the patient. By doing so, the residual limb RL is in a natural, weight bearing condition, which causes the skin and/or soft tissue to move slightly relative to the bone as compared to when the residual limb RL is not in a weight bearing condition. In this manner, the method produces a socket under conditions that more accurately reflect the actual conditions of use.

After actuating the vacuum pump for the time period, the portion of the limb can be removed from within the flexible liner 125 with the hardened socket material thereon. For example, the sealing member (e.g., sealing member 235) used to seal the top end portion of the flexible liner 125 to the upper portion of the limb of the patient can be removed or decoupled such that the portion of the limb can be removed from the flexible liner.

After the portion of the limb of the patient, with the hardened socket material thereon, has been removed from the flexible liner, the hardened socket material can be removed from the portion of the limb of the patient, thus resulting in a custom socket for attaching a prosthesis or an orthotic to a limb. In some embodiments, additional post-removal operations can be performed on the hardened socket material. For example, in some embodiments, the hardened socket material can be trimmed, can be coupled to a flange, or coupled to a locking mechanism for use in attaching the prosthesis or orthotic.

FIG. 3A illustrates another embodiment of a socket fabrication system 300 that can be used to fabricate a custom socket to attach a prosthesis or orthotic to a limb of a patient. The socket fabrication system 300 can include all the same components as described above for socket fabrication system 100 and/or the socket fabrication system 200 along with certain additional components as described herein. The socket fabrication system 300 includes a support stand 320 and a rigid outer cylinder 322 coupled to the support stand 320. The outer cylinder 322 includes an interior volume 323 in which a flexible liner 325 can be disposed, and multiple beads 330 are disposed within an interior region 326 of the flexible liner 325. A vacuum connector 332 has a first end 333 coupled to the cylinder 322 and a second end 334 coupled to a vacuum pump 336. Each of these components can be constructed the same as or similar to and perform the same as or similar function as like components described above for socket fabrication systems 100 and 200.

As shown in FIG. 3A, the support stand 320 includes an adjustment mechanism 338 configured to adjust a height position of the outer cylinder 322 to correspond to a height of the limb of the patient when the portion of the limb is placed in the flexible liner 325. Thus, with the limb of the patient within the rigid cylinder 322, the rigid outer cylinder 322 and/or the support stand 320 bears a portion of a weight of the patient during the fabricating process. In this manner, the system 300 can produce the socket with the portion of the limb in a mid-stance position, as described above. In this embodiment, the adjustment mechanism 338 includes a ratchet mechanism with a handle 337 coupled to an adjustment bar 339. Also shown in this embodiment, a filter 331 is coupled to the first end 333 of the vacuum connector 332 and used to prevent the beads 330 from moving out of the interior region 326 of the flexible liner 325 and into the vacuum connector 332. The filter 331 can be any suitable filter to prevent the beads 330 from being drawn out of the interior region 326 as a result of the vacuum pressure. For example, in some embodiments, the filter 331 can be a screen, mesh, or porous material having openings that are smaller than the nominal size of the beads 330.

As described herein, in use, a socket material and other fabrication components are first applied to the limb (residual limb RL in FIG. 3A) of the patient. As shown in FIGS. 3A and 3B, a protective liner 340 is applied about a portion of the limb of the patient and a socket material 341 is applied over the protective liner. A cover 342 is placed over the socket material 341. The cover 342 can be, for example, a thin plastic sheet of material wrapped around the socket material 341 on the limb of the patient. In some embodiments, the cover can be in the form of a sleeve.

As describe herein, the protective liner 340 can be any suitable protective liner for orthotic use. For example, in some embodiments, the protective liner is a gel liner. In some embodiments, the protective liner can be formed with, for example, a thermoplastic elastomer or a silicone material and can have suitable thickness and properties to function as the interface between the residual limb and the socket produced by the fabrication system 300. In some embodiments, the protective liner can include antioxidants that can be released from the protective liner over time (e.g., Vitamins A, B and C or any other antioxidants), skin conditioning agents (e.g., mineral oil or baby oil), or medicaments (e.g., to limit infection, promote healing of sores, etc.). In some embodiments, the protective liner can include multiple materials, such as an interior thermoplastic elastomer with an outer fabric layer. In some embodiments, the protective liner can include an attachment member, such as a pin (e.g., see pin 657 below), a stud or an internally threaded boss, to which a mating structure of an orthotic can be coupled.

The socket material 341 can be formed with multiple components as described herein. For example, the socket material 341 can include a fiber material that is impregnated with a hardenable material or the hardenable material can be otherwise coupled to the fiber material. For example, the socket material 341 can include, for example, a fiber material that is impregnated with a resin, such as polyurethane. In other embodiments the fiber material can be impregnated with a fiber-reinforced thermoplastic. The socket material 341 can be applied to the limb as a wrap or a “sock” that is placed over or wrapped about the protective liner 340. The socket material 341 can be wetted with, for example, water and the cover 342 is placed over the wetted socket material 341.

With the socket material 341 and other fabrication components (e.g., protective liner 340, cover 342) applied to the limb, the limb is placed into the interior region 326 of the flexible liner 325. For example, the patient while in a standing position on a support surface, can place the limb within the interior region 326 of the flexible liner 325 such that the limb is surrounded with the beads 330. A sealing member 335 is used to seal a top end of the flexible liner 325 to the limb of the patient as described herein. The sealing member 335 can be, for example, an elastomeric band, an adhesive liner, a tape, a belt, or other device that can be used to seal the flexible liner around the limb of the patient.

As described herein, the vacuum pump 336 can be actuated to create a vacuum within the interior region 325 of the flexible liner 325 such that the multiple beads 330 compress against the socket material 341 and the portion of the limb of the patient, conforming the socket material 341 to the limb. The vacuum 336 is actuated for a sufficient amount of time to allow the socket material 341 to cure and harden on the portion of the limb to form a custom socket to attach a prosthesis or orthotic to the limb.

After actuating the vacuum pump 336 for the desired time period, the sealing member 335 can be removed, and the portion of the limb can be removed from within the flexible liner 325 with the hardened socket material thereon. After the portion of the limb of the patient with the hardened socket material thereon, has been removed from the flexible liner 325, the hardened socket material can be removed from the portion of the limb of the patient, thus forming a custom socket for attaching a prosthesis or an orthotic to a limb.

In some embodiments, other components, such as an adapter plate can be attached to a bottom end of the hardened socket material that can be used to couple the hardened socket material to a prosthesis or to an orthotic. For example, the adapter plate can include a locking pin, a threaded pin, a pyramid adapter, a pyramid receiver adapter, a pylon clamp adapter, or any other adapter which can be removably coupled to (or interfaces with) the prosthesis or orthotic. In some embodiments, a one-way air expulsion valve can be attached to the adapter plate and to the bottom of the hardened socket material. Such valves can facilitate coupling the socket to the residual limb. Specifically, when the limb is inserted to the socket, the residual limb forms an airtight seal with the socket. The airtight seal is facilitated by producing the socket according to the methods described herein, which results in the inner surface of the socket conforming to the residual limb. The air expulsion valve can vent air from between the residual limb and the socket part during insertion of the limb, while also preventing the air from re-entering into the socket, thus maintaining the vacuum for a robust coupling of the socket to the residual limb.

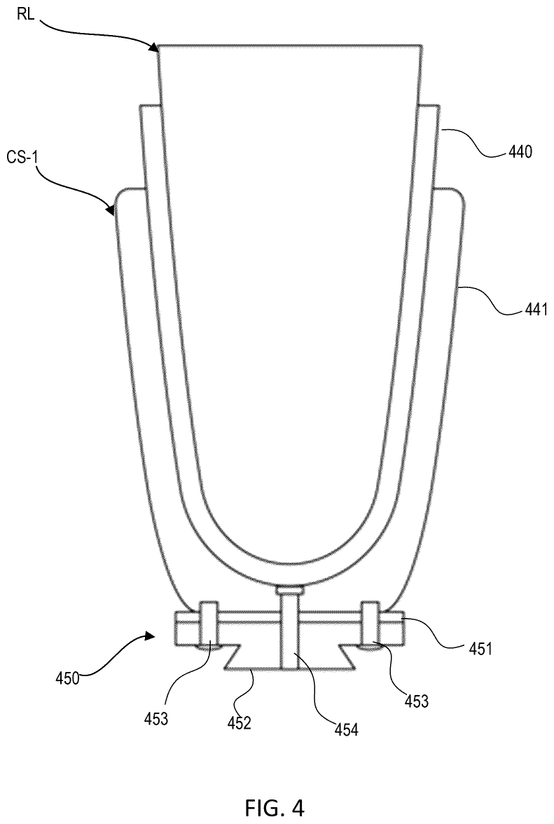

FIG. 4 illustrates an example custom socket CS-1 that can be formed using the socket fabrication system and methods described herein and that includes an adapter assembly coupled thereto. The custom socket CS-1 is formed from a socket material 441 that includes a fiber material and a hardenable material as described herein. The formed custom socket CS-1 is shown disposed on a residual limb RL with a protective liner 440 being between the residual limb RL and the inner surface of the custom socket CS-1.

After the socket CS-1 is formed using the socket fabrication systems and method described above, an adapter assembly 450 can be attached to the bottom end of the custom socket CS-1. In this example embodiment, the adapter assembly 450 includes a first adapter plate 451 and a second adapter plate 452 attached to the bottom end of the hardened socket material of the custom socket CS-1 with, for example, screws 453 that are secured through openings in the first and second adapter plates 451, 452 and directly into the hardened socket material of the custom socket CS-1. In other embodiments, the adapter plates can be coupled to the custom socket CS-1 by any suitable mechanism (e.g., adhesive) The adapter plates 451 and 452 can be standard adapter mechanisms used for such sockets (e.g., a pyramid adapter, a pyramid receiver adapter, or a boss within internally threaded bores). In addition, a vent line with a one-way air expulsion valve 454 can optionally be attached to the adapter plates 451 and 452 to allow for release of air between the internal region of the hardened socket material of the custom socket CS-1 and a region outside of the adapter plates 451. The vent line can be produced by drilling through the bottom end of the custom socket CS-1 and the one-way air expulsion valve can be mounted with one (or both) of the adapter plates 451, 452. The air expulsion valve can be any suitable check valve.

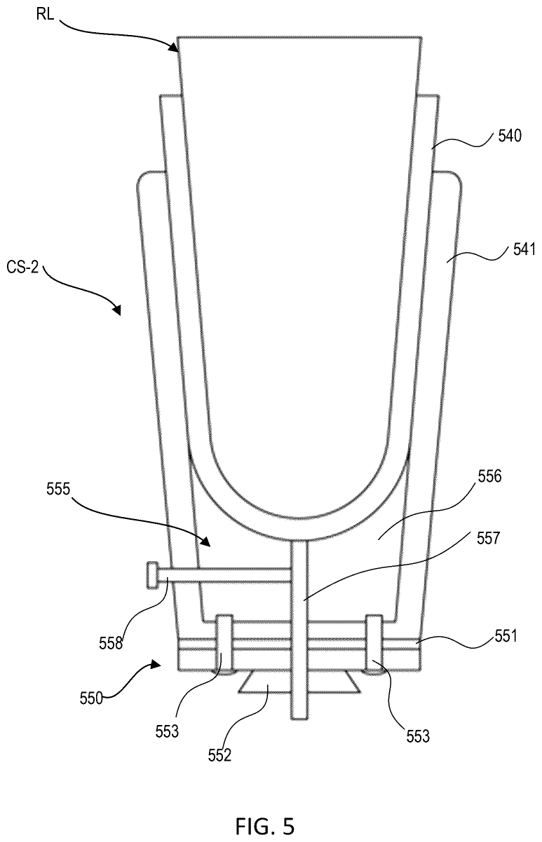

Although the custom socket CS-1 is shown as being coupled to the residual limb and/or protective liner via a vacuum coupling, in other embodiments, any of the sockets produced via the systems and methods herein can include any other suitable mechanism for being coupled to the patient's residual limb and/or prosthetic liner. For example, in some embodiments, it may be desirable to add a locking device to a custom socket during the fabrication process. FIG. 5 illustrates an example custom socket CS-2 formed with a custom socket fabrication system and method as described herein that includes such a lock mechanism. The custom socket CS-2 shown in FIG. 5 is formed from a socket material 541 that can include a fiber material and a hardenable material as described herein for other embodiments of a socket material. The formed custom socket CS-2 is shown disposed on a residual limb RL with a protective liner 540 (as described herein) being between the residual limb RL and the inner surface of the custom socket CS-2. The protective liner 540 includes an attachment pin 557 coupled to its distal end, and that is releasably locked within the lock device 555 of the custom socket CS-2, as described below. The attachment pin 557 can include any features that facilitate locking the pin 557 to the lock device 555, such as serrations or threads that engage with the release pin 558.

The custom socket CS-2 includes a lock device 555 that is added to the socket during the fabrication process. More specifically, prior to applying the socket material about the portion of the limb as described in the flowchart of FIG. 2 (see box 11), the lock device 555 is placed between the protective liner 540 and the socket material 541 at a lower end of the portion of the limb. As shown in FIG. 5, the lock device 555 includes a lock member 556 disposed between the protective liner and the socket material and a release pin 558. The lock member 556 defines an opening therethrough that receives the pin 557 from protective liner 540. The lock member 556 can be any suitable lock member and can include a distal surface that is molded or shaped to the end portion of the patient's limb. During the fabrication process, the pin 557 can be extended through (or beyond) the fiber material 541 that is applied about the portion of the limb. The custom socket CS-2 is otherwise fabricated in the same manner as described above using a vacuum force to conform the beads (e.g., beads 130, 230, 330) within the flexible liner (e.g., flexible liner 125, 225, 325) to the socket material 541 and to the limb of the patient and the lock member 556. The socket material 541 will thus conform around the lock member 556, the protective liner and thus the limb. The release pin 558 can be attached to the custom socket CS-2 after fabrication. Specifically, a lateral opening can be drilled or formed in the side of the custom socket CS-2 through which the release pin 558 can be movably disposed. In use, the release pin 558 can be used to releasably engage the pin 557 of the protective liner 540 to secure the custom socket CS-2 to the protective liner 540 and the patient's limb. For example, in some embodiments, the release pin 558 can be moveable within the socket CS-2 and can be spring-biased inward towards the pin 557. Thus, when the patient dons the socket CS-2, they will first pull the pin outward (against the spring force) and insert their residual limb into the socket CS-2 with the pin 557 being inserted into and/or through the lock member 556. The release pin 558 can then be released such that the spring force urges the end of the release pin 558 into locking engagement with the pin 557.

In some embodiments, rather than placing the lock device 555 between the socket material and the protective liner during the fabrication process, a “molding dummy” that is the same size as the lock device 555 is placed between the socket material and protective liner during the fabrication process and then replaced with the lock device 555 after the fabrication process. This can protect the lock device 555 from damage during the fabrication process.

In addition, as described above, after fabricating the custom socket CS-2, and removing the custom socket CS-2 from the limb, an adapter system 550 can be attached to the bottom end of the custom socket CS-2 in the same manner as described above for adapter system 450. More specifically, the adapter system 450 includes a first adapter plate 551 and a second adapter plate 552 attached to the bottom end of the custom socket CS-2 with, for example, screws 553 that are secured through openings in the first and second adapter plates 551 and 552 and directly into the custom socket CS-2. The adapter plates 551 and 552 can be standard adapter mechanisms used for such sockets. In use the adapter system 550 can be used to releasably couple the socket CS-2 to the prosthesis (not shown in FIG. 5).

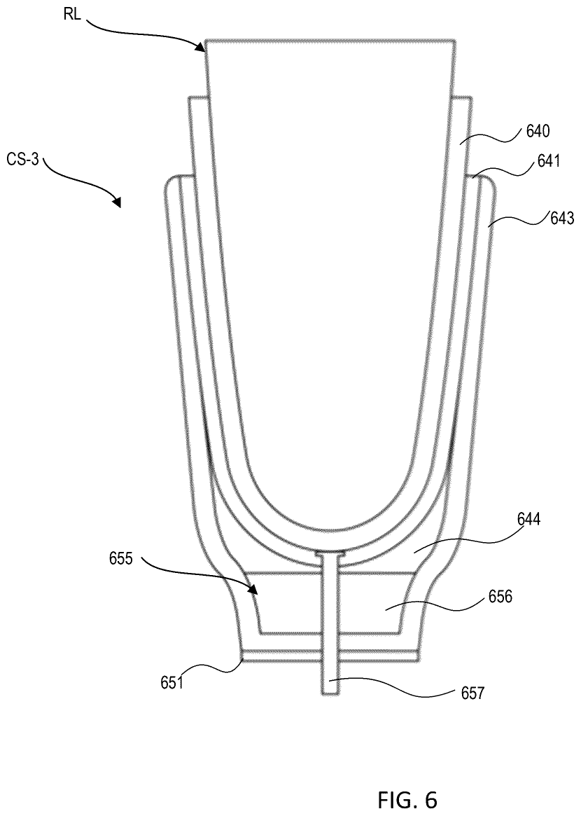

In some embodiments, the lock device for retaining the socket to the protective liner need not be in direct contact with the protective liner. For example, in some embodiments, a custom socket can be fabricated such that the socket material is formed about the entire distal end of the residual limb. In this manner, a lock device is not itself molded to or fit to the distal end of the residual limb, but is rather enclosed within the socket. Such configuration can provide a more even distribution of weight bearing pressure about the residual end, which can be beneficial for residual limbs having prominent features or where there is limited soft tissue padding that may be present at the distal end portion of the limb. For example, FIG. 6 illustrates another embodiment of a custom socket CS-3 for attaching an orthotic or prosthesis to a limb of a patient that can be fabricated using the socket fabrication systems and methods described herein. To accommodate the lock member 655 in a position that is not in direct contact with the protective liner 640, the custom socket CS-3 includes a double wall of fiber material. As shown in FIG. 6, the custom socket CS-3 is formed from two portions of socket material: a first socket material 641 and a second socket material 643. The first and second socket materials 641 and 643 can include the same materials as other socket materials described herein. The custom socket CS-3 is shown disposed on a residual limb RL with a protective liner 640 being between the residual limb RL and the inner surface of the first socket material 641. The protective liner 640 can be formed with the same materials as any of the protective liners described herein. The protective liner 640 includes an attachment pin 657 coupled to its distal end. The attachment pin 657 can be similar to the pin 557 described above, and can include any features that facilitate locking the pin 657 to the lock device 655 (described below), such as serrations or threads.

As shown in FIG. 6, the custom socket CS-3 also includes a lock device 655 that can be added to the socket during the fabrication process. The lock device 655 can be similar to or the same as the lock device 555 described above and includes a lock member 656 that can receive the pin 657. The lock device 655 can also include a release pin that is not shown in FIG. 6 or any other suitable features to retain the socket CS-3 about the residual limb.

To fabricate the double walled socket of FIG. 6, the process described above is performed a first time to fabricate the first socket material 641 (i.e., the inner most portion of the custom socket CS-3. Then the second layer of socket material 643 is added to (or wrapped about) the hardened first socket material 641 and the lock device 655 and the process is performed a second time. More specifically, as shown in FIG. 6, the protective liner 640 is first applied about a portion of the limb of the patient and then the first socket material 641 is applied over the protective liner 640. The first socket material 641 is wetted with, for example, water, and a cover (not shown, but which can be similar to the cover 342) is placed over the first socket material 641. The cover can be, for example, a thin plastic sheet of material wrapped around the first socket material 641 on the limb of the patient. In some embodiments, the cover can be in the form of a sleeve. In this embodiment, the locking pin 657 is extended from the protective liner 640 and through the first socket material 641.

With the socket material applied to the limb, the limb is placed into the interior region of the flexible liner (not shown, but which can be similar to the flexible liner 325). For example, the patient while in a standing position on a support surface, can place the limb within the interior region of the flexible liner such that the limb is surrounded with the beads (e.g., beads 130, 230, 330) within the inter region of the flexible liner. A sealing member (not shown) is used to seal a top end of the flexible liner to the limb of the patient as described herein. The sealing member can be, for example, an elastomeric band, an adhesive liner, a tape, a belt, or other device that can be used to seal the flexible liner around the limb of the patient.

As described herein, a vacuum pump (e.g., 236, 336) can be actuated to create a vacuum within the interior region of the flexible liner such that the multiple beads compress against the first socket material 641 and the portion of the limb of the patient, conforming the first socket material 641 to the limb. The vacuum pump is actuated for a sufficient amount of time to allow the first socket material 641 to cure and harden on the portion of the limb.

After actuating the vacuum pump (e.g., pump 236, 336) for the desired time period, the sealing member can be removed, and the portion of the limb can be removed from within the flexible liner with the first layer of hardened socket material 641 thereon. To form the second layer of the socket, the second socket material 643 is then applied over the hardened first socket material. In this embodiment, prior to applying the second socket material 643, a filler component 644 (such as, for example, a foam material) and the lock member 656 of the lock device 655 are placed at the bottom of the hardened socket material 641 with the pin 657 extending through both.

The second socket material 643 is wetted to activate the hardenable material and the limb with the second socket material 643 thereon is again placed into the interior region of the flexible liner. For example, the patient while in a standing position on a support surface, can place the limb within the interior region of the flexible liner such that the limb is surrounded with the beads (e.g., beads 130, 230, 330) within the inter region. The sealing member (not shown) is again used to seal a top end of the flexible liner to the limb of the patient as described herein.

The vacuum pump (e.g., pump 236, 336) can be actuated again to create a vacuum within the interior region of the flexible liner such that the multiple beads compress against the second socket material 643, conforming the second socket material 641 to the first hardened socket material 641, the filler component 644 and/or the lock device 655. The vacuum is actuated for a sufficient amount of time to allow the second socket material 643 to cure and harden about the first hardened fiber material 641.

After actuating the vacuum pump for the desired time period, the sealing member can be removed, and the portion of the limb can be removed from within the flexible liner with the first and second hardened socket materials thereon. The hardened socket materials are now hardened into a single socket that can be removed from the portion of the limb of the patient, thus forming the custom double walled socket CS-3 for attaching a prosthesis or an orthotic to the limb.

As with previous embodiments, after fabricating the custom socket CS-3, and removing the custom socket CS-3 from the limb, an adapter system can be attached to the bottom end of the custom socket CS-3 in the same manner as described above for adapter system 450. In the embodiment of FIG. 6, a single adapter plate 651 is attached to the end of the socket It should be understood that other adapter systems and devices can be attached to the custom socket CS-3 such as the adapter systems 450 and 550 described above.

FIGS. 7 and 8 each illustrate an example coupling method for coupling a flexible liner (e.g., the flexible liner 325) to a vacuum connector in a socket fabrication system described herein. FIG. 7 illustrates a flexible liner assembly 760 that includes a flexible liner 725, a filter 731 and a barbed fitting 729. As described herein, the flexible liner 725 can be constructed with, for example a rubber, plastic or other flexible material. The flexible liner 725 defines an interior region 726 and a limb opening 727 at a top end of the flexible liner 725. The flexible liner assembly 760 can be used in a socket fabrication system as described herein and placed within a rigid cylinder of the system, and filled with beads (e.g., beads 130, 230, 330). In this embodiment, the barbed fitting 729 can be coupled to the flexible liner 725 with, for example, a plastic weld, adhesive or other suitable coupling method. The barbed fitting 729 can be placed though an opening in the bottom of the outer cylinder (e.g., the outer cylinder 322) and be coupled to a vacuum connector (e.g., vacuum connector 132, 232, 332) to fluidically couple the flexible liner 725 to a vacuum pump (e.g., vacuum pump 236, 336).

FIG. 8 illustrates a portion of a flexible liner assembly 860 and a portion of a rigid cylinder 822 that can be used in a socket fabrication system as described herein. The flexible liner assembly 860 includes a flexible liner 825 and a filter 831. The flexible liner 825 defines an interior region 826 and a limb opening 827 at a top end of the flexible liner 825. The filter 831 is part of a threaded plug 846 that can be threadably coupled to a mating threaded flange 847 of the rigid cylinder 822. The flexible liner 825 can include an opening at the bottom end that forms a flange portion 848 that can be captured between the threaded plug 846 and the threaded flange 847 of the cylinder 822. The flange 847 of the cylinder 822 can be coupled to a vacuum connector (e.g., 132, 232, 332) to fluidically couple the flexible liner 825 to a vacuum pump (e.g., 236, 336).

FIG. 9 is a perspective view of another embodiment of a socket fabrication system 900 showing the system 900 in use to fabricate a custom socket to attach a prosthesis or orthotic to a limb (e.g., residual limb RL in FIG. 9) of a patient. The socket fabrication system 900 can include all the same components as described above for socket fabrication systems 100, 200, 300 including a support stand 920 and a rigid outer cylinder 922 coupled to the support stand 920, a flexible liner 925, a vacuum connector 932 and a vacuum pump 936. The outer cylinder 922 includes an interior volume (not shown) in which the flexible liner 925 is disposed, and multiple beads (not shown) are disposed within an interior region of the flexible liner 925. The vacuum connector 932 has a first end coupled to the cylinder 922 and a second end coupled to the vacuum pump 936. A sealing member 935 is used to couple a top end of the flexible liner 925 to the limb such that here is no direct coupling of the top portion of the flexible liner 925 to the outer cylinder 922. of the patient as described herein. Each of these components can be constructed the same as or similar to and perform the same as or similar function as like components described above for any of the socket fabrication systems described herein.

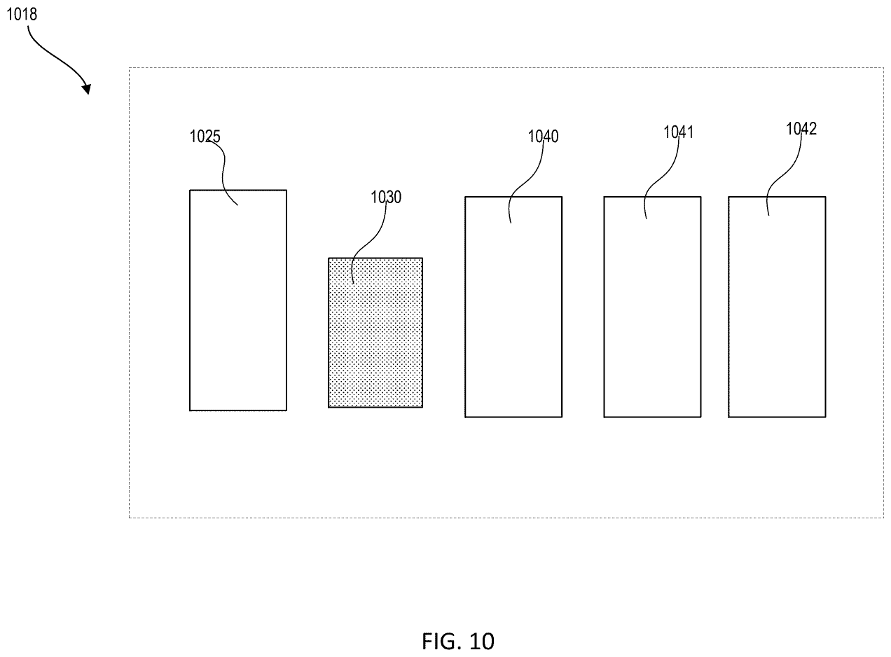

FIG. 10 illustrates a kit for use in fabricating a custom socket for attaching a prosthesis or an orthotic to a limb of a patient. The kit 1018 can be used in any embodiment of a socket fabrication system described herein. The kit 1018 can include any of the following components: a flexible liner 1025, a plurality of beads 1030, a protective liner 1040, a socket material component 1041 (e.g., a fiber material and a hardenable material), and a cover 1042. The flexible liner 1025 can be any of the flexible liners described herein. For example, the flexible liner 1025 can include a feature and/or coupler to couple the flexible liner 1025 to a vacuum pump via a vacuum connector. In some embodiments, the coupler can be similar to the fitting 729. In some embodiments, the flexible liner 1025 can include an opening at a bottom end to couple to a threaded flange of a cylinder as described for flexible liner 825. The protective liner 1040 can be formed with the same materials and construction as described herein and is configured to be placed on the end portion of a limb of a patient. The socket material component 1041 is configured to be applied over the protective liner 1040. The socket material component 1041 can be the same as any of the socket materials described herein. The cover 1042 is configured to be placed over the socket material component 1041. In some embodiments, the kit can also include one or more adapter plates configured to be coupled to the socket produced with the socket fabrication system and to one of the prosthetic and the orthotic. The adapter plate(s) can be included in an adapter assembly (e.g., 450, 550) as described herein. In some embodiments, the kit includes a lock device including a lock member and a pin, the lock device is configured to be coupled between the protective liner 1040 and the socket material component 1041 during fabrication of the socket using the socket fabrication system.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. Additionally certain events may be performed concurrently in parallel processes when possible, as well as performed sequentially. While the embodiments have been particularly shown and described, it will be understood that various changes in form and details may be made. Although various embodiments have been described as having particular features and/or combinations of components, other embodiments are possible having a combination of any features and/or components from any of embodiments where appropriate. Any of the components and sub-components described herein can be included in any of the embodiments unless mutually exclusive.

1. An apparatus, comprising:
a support stand;
a rigid outer cylinder coupled to the support stand, the outer cylinder defining an interior volume and an opening that provides access to the interior volume;
a flexible liner configured to be disposed inside the interior volume of the outer cylinder, the flexible liner defining an interior region and a limb opening at a top end of the flexible liner, the limb opening of the flexible liner configured to receive a portion of a limb of a patient with a socket material disposed thereon such that the portion of the limb with the socket material thereon is positioned inside the interior region of the flexible liner, and the top end of the flexible liner configured to be sealed around an upper portion of the limb outside of the cylinder;
a plurality of beads disposed within the interior region of the flexible liner; and
a vacuum connector having a first end and a second end, the first end coupled to a bottom end of the outer cylinder and in fluid communication with the interior region of the flexible liner, the second end configured to be coupled to a vacuum pump, the vacuum pump configured to create a vacuum within the flexible liner such that the plurality of beads compress against the socket material and the portion of the limb when the portion of the limb is positioned in the interior region of the flexible liner and the top end of the flexible liner is sealed to the limb.

2. The apparatus of claim 1, wherein the support stand includes an adjustment mechanism configured to adjust a height position of the cylinder.

3. The apparatus of claim 1, further comprising:
a filter coupled to the first end of the vacuum connector to prevent the plurality of beads from moving out of the interior region of the flexible liner.

4. The apparatus of claim 1, further comprising:
a sealing member configured to seal the top end of the flexible liner to the upper portion of the limb.

5. The apparatus of claim 1, wherein the sealing member includes a strap.

6. The apparatus of claim 1, wherein the sealing member includes a tape.

7. A method of fabricating a socket for attaching a prosthesis or an orthotic to a limb, comprising:
applying a socket material to a portion of a limb of a patient, the socket material to be used in the formation of the socket;
after the applying, placing the portion of the limb into an interior region of a flexible liner of a socket fabrication system, the socket fabrication system including a rigid outer cylinder defining an interior volume in which the flexible liner is configured to be disposed and a plurality of beads within the interior region of the flexible liner;
sealing a top end portion of the flexible liner to an upper portion of the limb outside of the cylinder; and
producing a vacuum within the interior region of the flexible liner with the patient in a standing position and the rigid outer cylinder bearing a portion of a weight of the patient, the vacuum causing the plurality of beads to compress against the socket material such that the socket material conforms to the portion of the limb.

8. The method of claim 7, wherein the socket fabrication system includes a support stand including a height adjustment mechanism, the method further comprising:
prior to the producing a vacuum, adjusting a height of the rigid outer cylinder to a height associated with a height of the portion of the limb of the patient when the portion of the limb is placed into the interior region of the flexible liner.

9. The method of claim 7, further comprising:
prior to applying the socket material, placing a protective liner about the portion of the limb;
wetting the socket material; and
placing a cover over the socket material.

10. The method of claim 9, wherein:
the producing the vacuum includes actuating a vacuum pump coupled to a bottom end portion of the rigid cylinder for a time period, the vacuum pump coupled to a vacuum hose in fluid communication with the interior region of the flexible liner.

11. The method of claim 10, wherein:
the actuating the vacuum pump for the time period includes actuating the vacuum pump for the time period sufficient for the socket material to harden forming a hardened socket material, the method further comprising:
after the actuating the vacuum pump, removing the end portion of the limb from within the flexible liner with the hardened socket material thereon.

12. The method of claim 9, wherein the producing the vacuum includes producing the vacuum for a time period sufficient for the socket material to harden forming a hardened socket material, the method further comprising:
after the producing the vacuum, removing the end portion of the limb from within the flexible liner with the hardened socket material thereon.

13. The method of claim 12, further comprising:
removing the hardened socket material from the end portion of the limb of the patient;
attaching an adapter plate to the bottom end of the hardened socket material, the adapter plate configured to be coupled to one of the prosthesis or the orthotic.

14. The method of claim 13, further comprising:
attaching a one-way air expulsion valve to the adapter plate and to the bottom of the hardened socket material.

15. The method of claim 9, further comprising:
prior to applying the socket material, placing a lock device between the protective liner and the socket material at a lower end of the portion of the limb.

16. The method of claim 15, wherein the producing the vacuum includes producing the vacuum for a time period sufficient for the socket material to harden forming a hardened socket material, the method further comprising:
after the producing the vacuum, removing the portion of the limb from within the flexible liner with the hardened socket material and the lock device thereon.

17. (canceled)

18. The method of claim 9, further comprising:
prior to applying the socket material, placing a first end of a lock pin between the protective liner and the socket material at a bottom end portion of the limb such that a second end of the lock pin extends outside of the socket material.

19-23. (canceled)

24. A kit for fabricating a socket for attaching a prosthesis or an orthotic to a limb using a socket fabrication system, comprising:
a flexible liner configured to be disposed inside an interior volume of a rigid outer cylinder of the socket fabrication system, the flexible liner defining an interior region configured to receive an end portion of a limb of a patient with a socket material disposed thereon, the flexible liner including a coupling configured to be coupled to a vacuum pump via a vacuum hose to place the vacuum pump in fluid communication with the interior region of the flexible liner;
a plurality of beads configured to be disposed within the interior region of the flexible liner;
a protective liner configured to be placed on the end portion of the limb of the patient;
a socket material configured to be applied over the protective liner; and
a cover configured to be placed over the socket material.

25. The kit of claim 24, further comprising:
an adapter plate configured to be coupled to the socket fabricated with the socket fabrication system and to one of the prosthetic and the orthotic.

26. The kit of claim 24, further comprising:
a lock device including a lock member and a pin, the lock device configured to be coupled between the protective liner and the socket material during fabrication of the socket using the socket fabrication system.
